(19) United States
(12) Patent Application Publication
(10) Pub. No.: US 2011/0046242 A1
(43) Pub. Date: Feb. 24, 2011

(54) Film Forming, Silicone Containing Compositions

(76) Inventors: Jean-Luc Garaud, Comines (FR); Guillaume Kergosien, Chaville (FR); Xavier Thomas, Famars (FR)

Correspondence Address: DOW CORNING CORPORATION CO1232, 2200 W. SALZBURG ROAD, P.O. BOX 994, MIDLAND, MI, 48686-0994, US

(21) Appl. No.: 12/863,006

(22) PCT Filed: Jan. 16, 2009
(86) PCT No.: PCT/EP2009/000237

§ 371 Date: Aug. 18, 2010

(30) Foreign Application Priority Data
Jan. 17, 2008 (EP) 08305005.4

(51) Int. Cl. A61K 47/34

(52) U.S. Cl. 514/772.3

(57) ABSTRACT

The invention relates to silicone containing compositions able to form adhesive films on substrates, which typically comprises a curable silicone formulation containing: (a) a polyorganosiloxane polymer having at least two functional SiVi groups per molecule, each SiVi group containing an alkenyl functionality directly bonded to a silicon atom; (b) a crosslinker polyorganosiloxane compound having at least 3 Si-bonded hydrogen groups or SiH groups per molecule; (c) a chain extender compound which is a telechelic polyorganosiloxane having terminal SiH groups; (d) a hydrosilylation catalyst for the reaction of SiH groups with SiVi groups; (e) with RHV>1.5 wherein RHV is the ratio of the number of SiH moles in (b) and (c) to the number of SiVi moles in (a) and (d), and 0<RHC<0.7 wherein RHC is the ratio of the number of SiH moles in (c) to the number of SiH moles in (b) and (c). Such formulation can cure quickly and can provide good balance between adhesion and tack.

This invention relates to silicone containing compositions able to form adhesive films on substrates, especially on biological substrates, which can be vegetal or animal, for example human skin or vegetal crust.

Such film forming, silicone containing compositions can be useful in the fields of cosmetics, pharmaceutical and medical applications.

A cosmetic product, as defined under Article 1 of the European Directive 76/768/EEC dated 27 Jul. 1976, means any substance or preparation intended to be placed in contact with the various external parts of the human body (epidermis, hair system, nails, lips and external genital organs) or with the teeth and the mucous membranes of the oral cavity with a view exclusively or mainly to cleaning them, perfuming them, changing their appearance and/or correcting body odours and/or protecting them or keeping them in good condition.

Delimiting the field of pharmaceuticals from cosmetics not only follows from the detailed definition of cosmetic products, which refers to both their areas of application and to the purposes of their use: some products may fall under the definition of cosmetic product but are exclusively intended to protect from diseases and integrity alteration, and to restore integrity, such products are then regulated products such as pharmaceutical products or medical devices and, moreover, products containing substances or preparations intended to be ingested, inhaled, injected or implanted in the human body do not come under the field of cosmetics. Products such as skin barrier, bandage, gauze or wound dressing products also come under the pharmaceutical area.

A pharmaceutical or medical product typically contains a therapeutic active agent X having a pharmaceutical or medical action whereas the vehicle Y may be similar to the one of a cosmetics product.

Many formulations for forming films are known in the medical and pharmaceutical art. These include, for example, ointments, salves, creams, lotions, gels, elastomers and the like.

For example, WO 01/96450 describes one-part formulations which rapidly cure on exposure to moisture and are useful for forming films in personal and healthcare applications. The formulations comprise an alkylene trialkoxy terminated siloxane, a catalyst, a diluent and optionally an alkoxysilane and/or a filler. The formulation reacts in presence of moisture to cure by a condensation reaction.

Formulations for use in cosmetic or medial applications, and in general for use on living bodies should cure at room temperature for example between 20 and 30C, or at the temperature of the living body.

WO 2000/74738 describes use of Room Temperature Curing silicone compositions for wound dressing. The compositions contain a crosslinkable polysiloxane, a crosslinking agent and a catalyst, and are applied to a wound to treat it.

WO 2004/108175 also describes a preparation for application to a wounded skin to protect it. The preparation preferably consists of an addition-curing RTV (Room Temperature Vulcanization) silicone system.

Compositions formulated to enable to load and release pharmaceutical actives from a cured membrane are also known in the art.

In one of its aspects, the present invention relates to a method of making topical active delivery compositions and to the use of silicone based formulations that rapidly solidify for forming films onto skin, mucosa or wound tissue to deliver pharmaceutical active molecules.

Many compositions for the topical delivery of pharmaceuticals are known in the art. These include, for example, mucosal dosage forms, transdermal delivery systems, dermatological and subcutaneous therapeutic treatments, medicated wound dressings and the like.

Some of the known drug delivery compositions use silicone based materials as matrices or membranes through which pharmaceutical agents are able to diffuse onto and into the body to deliver locally or systemically the therapeutic effect. Silicone based materials are desirable in these compositions since they form films onto the body and allow for sustained or controlled release of actives.

EP 0322118 describes siloxane gels which can be used for gel dressings and in medical prostheses. The gels are made from compositions comprising (A) alkenyl-containing polydiorganosiloxanes; (B) hydrosilicon compounds having at least 3 Si—H groups; (C) SiH end-blocked polydiorganosiloxanes and (D) a catalyst. The compositions must have a ratio (RHAlk) of SiH:Si-Alkenyl of from 1:1 to 20:1, the percentage of silicon-bonded H atoms provided by (C) not being less than 81.36−(3.6×RHAlk) and having a value of from 10% to 90%.

EP 465744 describes a sustained release formulation comprising an agent (A) which is to be released and a vehicle (B) therefore. The vehicle comprises a hydrophilic component and a curable silicone composition containing a polysiloxane having alkyhydrogen units, a polysiloxane having unsaturated groups and a platinum or rhodium catalyst. The formulation may be applied to the human or animal body or a cavity in the latter to cure in situ to give a dressing capable of sustained release of the therapeutic or diagnostic agent (A) to the body.

EP 0865787 describes another silicone based material suitable for in situ applications e.g. on a human or animal body. EP 0865787 describes a method of making a controlled release composition comprising preparing a sprayable formulation and spraying the sprayable formulation onto the desired site, wherein said spraying causes mixing of the sprayable formulation and wherein said mixed sprayable formulation cures in situ on the desired site to form the controlled release composition.

WO2008/057155, published 15 May 2008, describes “silicone gel forming compositions that have an average RHAlk of 0.7 to 1.5, typically 0.8 o 0.95 and with an average RHCE of 0.4 to 1, typically 0.8 to 0.95 and the silicone gels produced by curing the gel forming composition. These gels are suitable for temporarily adhering a medical device to a biological substrate such as skin.”

US2003/0214051 describes a semiconductor package comprising a semi conductor wafer having an active surface comprising at least one integrated circuit, wherein each integrated circuit has a plurality of bond pads, and at least one cured silicone member covering at least a portion of the active surface. The silicone member is a polymer obtained by heating, typically at 150C, a silicone composition to let it undergo a hydrosilylation reaction.

Patent Publication JP08-134427 describes a hardenable pressure-sensitive adhesive composition able to realise a bonding between 2 surfaces, like an adhesive tape, wallpaper, label etc. On the contrary, the present invention relates to a film forming composition for which the composition should adhere to a biological substrate on one side but forming a film preferably not tacky on the other side.

WO2007/071706 describes a cosmetic process for coating keratin materials, which consists in applying to a keratin material, in particular the skin, the lips, the eyelashes, the eyebrows or the nails, at least one compound A and at least one compound B, at least one of the compounds A and B being a silicone compound, the said compound A and B being capable of reacting together via a hydrosilylation reaction a condensation reaction or crosslinking reaction in the presence of a peroxide.

A system comprising silicone compounds that polymerize in situ permits to obtain cosmetic compositions, which may exhibit one or more advantageous properties such as good transfer resistance, staying power over time in particular resistance to water and rubbing, a comfortable deposit on the skin and good biocompatibility of the silicone with the skin.

It is still desired to provide improved methods for formulating in situ film forming compositions which combine a fast cure, a good adhesion to substrate, a low tack surface and suitable compatibility with pharmaceutical or cosmetic products.

### BRIEF DESCRIPTION OF THE INVENTION

We have now discovered that silicone containing formulations with certain amounts of components are able to meet the objectives of combining fast cure, good adhesion and low tack.

We have also discovered a more suitable method of making a controlled release composition which can form in situ a non-tacky and elastic film.

In one of its aspects, the invention provides a composition comprising a curable silicone formulation containing:
(a) a polyorganosiloxane polymer having at least two A groups per molecule,
(b) a crosslinker polyorganosiloxane compound having at least 3 B groups per molecule,
(c) a chain extender compound which is a telechelic (carrying functional end groups) polyorganosiloxane having terminal B groups, the groups A and B being either a functional group containing an alkenyl functionality, which functional group is directly bonded to a silicon atom (herein after called “SiVi” group) or a Si-bonded hydrogen group (hereinafter called “SiH” group), provided that when A is SiVi, B is SiH and when A is SiH, B is SiVi.
(d) a hydrosilylation catalyst for the reaction of SiH groups with SiVi groups,
(e) wherein the formulation is such that:
   (1) RHV>1.5 wherein RHV is the ratio of the number of B moles in (b) and (c) with respect to the number of A moles in (a) and (d), and
   (2) 0<RHC<0.7 wherein RHC is the ratio of the number of B moles in (c) with respect to the number of B moles in (b) and (c).

A formulation meeting these requirements of RHV and RHC is able to cure quickly as a film on a substrate and can provide good balance between adhesion and tackiness requirements; the film can show good adhesion to the substrate while the surface opposite to the substrate shows low tack.

It is not important whether the silicon-bonded hydrogen group or the alkenyl group is on component (a) or (b) and (c) provided one is solely found on component (a) and the other is predominantly found on component (b) and (c). However, commercial SiH compounds with short chain and terminal SiH are readily commercially available while long chain SiH compounds are more difficult to find on the market. Therefore, it is preferred that the reactive group A is a vinyl or other alkenyl-containing functional group directly bonded to a silicon atom and group B is a hydrogenosiloxyl SiH group. In the following description, component (a) is described as of the SiVi type while (b) and (c) are of SiH type but as is apparent from the above, these reactive groups can be interchanged.

Preferably, RHV>2.5, more preferably RHV>3.

The present invention provides in one of its aspects a controlled release composition for medical or pharmaceutical use comprising a spreadable formulation containing an active agent X and a vehicle Y, said vehicle comprising a curable silicone formulation containing:
(a) a polydiorganosiloxane having at least two silicon-bonded alkenyl groups per molecule, with preferably the remaining silicon-bonded organic groups being selected from alkyl and aryl groups, said polydiorganosiloxane having a viscosity at 25° C. of from 3 mm²/s to 100,000 mm²/s,
(b) a preferably linear hydrosilicon compound having at least 3 silicon-bonded hydrogen atoms per molecule, which preferably consists essentially of RHSiO-groups, R2ZSiO-groups and optionally R2SiO-groups and having a viscosity at 25° C. of no more than 1000 mm²/s, wherein R denotes an alkyl or aryl group having no more than 8 carbon atoms, and Z denotes H or R,
(c) a diorganohydrogensiloxy-terminated polydiorganosiloxane, wherein preferably the organic substituents are alkyl or aryl groups having no more than 8 carbon atoms,
(d) a hydrosilylation catalyst for the reaction of SiH groups with Si-Alkenyl groups.

The formulation of the invention is characterised in that the vehicle Y is formulated in order to obtain RHV>1.5, preferably >2.5 wherein RHV is the ratio of the number of SiH moles in (b) and (c) with respect to the number of Si-Alkenyl moles in (a) and (d), and 0<RHC<0.7, preferably RHC<0.5, wherein RHC is the ratio of the number of SiH moles in (c) with respect to the number of SiH moles in (b) and (c).

A RHV below 1 or below 1.5 may provide pressure sensitive adhesive properties on the surface of the cured film, for example, rendering it tacky to touch. In addition, the reaction speed is lower.

A RHC of 0.7 or more may provide tackiness and/or low cohesive strength of the cured film.

### DETAILED DESCRIPTION OF THE INVENTION

The component materials of vehicle Y can either be separated into a plurality of containers to inhibit curing prior to spreading or can be packaged into a unique container wherein the hydrosilylation catalyst (d) is temporarily inhibited, wherein the temporary inhibition is obtained by encapsulating the hydrosilylation catalyst (d) or by adding transient inhibitors.

The formulation of the invention can be applied by spreading the formulation onto the desired site, wherein said spreading can cause mixing the formulation or the mixing of the formulation is achieved prior spreading the formulation.

The activation of the catalyst can occur prior to spreading the formulation, or can be caused by spreading the formulation, or can be caused by mixing the formulation, or can occur after spreading the formulation.

After spreading, the formulation cures in situ on the desired site to form the sustained release composition. The resultant sustained release composition is then capable of delivering a pharmaceutical or biological active X at a controlled rate.

The active agent X used in the present invention can comprise any solid or liquid material which can be bound in the sustained release composition and subsequently released at the desired rate. The active agent X should also not interfere with the curing of the silicone formulation to an unacceptable extent. Suitable active agents X include cosmetics and therapeutic or diagnostic materials.

The invention is especially applicable to those therapeutic and diagnostic active agents X which benefit from topical delivery over a period of time at a sustained rate. For example, it is known that for certain drugs it is desirable to have the quantity of drug in the animal body continuously remain within a therapeutic window. By tailoring a formulation according to the invention, it is possible to provide sustained release compositions which deliver the drugs at rates which keep their concentrations in the body within their therapeutic windows.

Therapeutic active agents X which may be employed include, for example, anti acne agent, antibiotic, antiseptic, antifungal, antibacterial, antimicrobial, biocides, anti-inflammatory, astringents, hormones, anticancer agents, smoking cessation compositions, 30 cardiovascular, histamine blocker, bronchodilator, analgesic, antiarrythmic, antihistamine, alpha-I blocker, beta blocker, ACE inhibitor, diuretic, antiaggregant, sedative, tranquillizer, anticonvulsant, anticoagulant agents, vitamins, antiaging agents, agents for treating gastric and duodenal ulcers, anticellulites, proteolytic enzymes, healing factors, cell growth nutrients, peptides and others. Specific examples of suitable therapeutic active agents include penicillins, cephalosporins, tetracyclines, macrolides, epinephne, amphetamines, aspirin, acetaminophen, barbiturates, catecholamines, benzodiazepine, thiopental, codeine, morphine, 5 procaine, lidocaine, benzocaine, sulphonamides, ticonazole, perbuterol, furosamide, prazosin, prostaglandins, salbutamol, indomethicane, diclofenac, glafenine, dipyridamole, theophylline and retinol.

Specific examples of suitable therapeutic active agents X include penicillins, cephalosporins, tetracyclines, macrolides, epinephrine, amphetamines, aspirin, barbiturates, catecholamines, benzodiazepine, thiopental, codeine, morphine, procaine, lidocaine, sulphonamides, ticonazole, perbuterol, furosamide, prazosin, prostaglandins, salbutamol, indomethicane, diclofenac, glafenine, dipyridamole, and theophylline.

In addition to the therapeutic or diagnostic materials, active agents X could be cosmetics such as perfumes, deodorants, pigments, anti-perspirant compounds, waxes, gelling agent or another silicone compound able to provide a soft and silky touch, or the like. Suitable cosmetics are known to those skilled in the art.

The proportion of the active agent X employed in the present invention is chosen in accordance with the concentration of the active agent X required in the sustained release composition to deliver the dosage required at the proposed delivery rate. This may vary within a wide range such as from 0.001 weight percent to about 70 weight percent, preferably 0.01 weight percent to 20 weight percent of the final sustained release composition.

The vehicle Y used in the present invention comprises silicones (‘polysiloxanes’) or silicone-based materials which cure to form binder matrices for the other components (e.g. the active agent X of the invention (i.e., they contain or entrap such components).

The polysiloxanes used herein comprise those which have silicon-bonded hydrogen atoms (b) and (c), in combination with those that have silicon-bonded unsaturated hydrocarbon groups (a). These polysiloxanes undergo a hydrosilylation reaction in the presence of a catalyst (d) to yield chain extended or cross-linked elastomeric silicone films.

Suitable polysiloxanes (b) having silicon bonded hydrogen (e.g. Si—H) include those having units according to the general formula RpHSiO(3-p/2) in which each R represents a monovalent hydrocarbon group containing 1 to 20 carbon atoms, such as alkyl (e.g., methyl, ethyl, propyl or butyl) or phenyl groups and p is 0, 1, or 2. It is preferred that each R represents a methyl group. It is also preferred that the terminal groups have the formula R3SiO1/2 where each R represents a methyl group.

The polysiloxanes (b) having silicon bonded hydrogen may include those forming cyclics, for example pentamethylcyclopentasiloxane (D5H).

The polysiloxanes (b) having silicon bonded hydrogen atoms may alternatively be copolymers comprising, for example, units RnSiO(4-n/2) in which R is as referred to above, and n is 0, 1, 2 or 3.

The polysiloxanes (b) having silicon bonded hydrogen may alternatively include a siloxane (silicone) resin structure with silicon bonded hydrogen. The siloxane resin structure may comprise R₃SiO_{1/2} units (M units) and SiO_{4/2} units (Q units) wherein each R is independently a linear, branched or cyclic hydrocarbon group having 1-20 carbon atoms. R can be unsubstituted or substituted with halogen atoms. Each R can be identical or different, as desired. The hydrocarbon group of R can be exemplified by alkyl groups such as methyl, ethyl, propyl, butyl, hexyl, octyl, vinyl, hexenyl, 3,3,3-trifluoropropyl, chloromethyl, and decyl, cycloaliphatic groups such as cyclohexyl, aryl groups such as phenyl, tolyl, and xylyl, chlorophenyl, and aralkyl groups such as benzyl, styryl and alpha-methylstyryl. The resin can also contain triorganosiloxy units (T units), for example 0.5 to 1 triorganosiloxy group for every SiO_{4/2} unit, alternatively 0.6 to 0.9 triorganosiloxy group for every SiO_{4/2} unit.

The siloxane resin structure may comprise RSiO_{3/2} units (also known as T units) in which the groups R, which can be different in different siloxane units, are selected from hydroxyl, hydrocarbon, substituted hydrocarbon, hydrocarbonoxy and substituted hydrocarbonoxy groups. The silicone resin optionally also comprises R₂SiO_{2/2} units (D units) and/or R₃SiO_{1/2} units (M units), in which each R is defined as above. The hydrocarbon and hydrocarbonoxy groups each preferably contain 1 to 20, more preferably 1 to 8, carbon atoms.

It should be noted that more than 1 resin could be included in the present invention. In this case, at least one of the resins can have some silanol content which is deemed to be favourable to adhesion properties or one could have the silanol capped so that there is substantially no silanol present. It should also be noted that other resins can be also added to the composition of this invention. For example, organic resins could be added if desired. In one embodiment, for example, a vinyl-functional resin can be added.

Resins comprising R₃SiO_{1/2} units and SiO_{4/2} units are well known in the art. These copolymers are described, for example, in U.S. Pat. Nos. 3,936,582, 2,676,182, and 2,857,356. The resinous copolymers can be prepared by cohydrolysis of a mixture of silanes having four hydrolyzable groups, e.g., silicon tetrachloride, and triorganosilanes having one hydrolyzable group, e.g., trimethylchlorosilane, in the proper ratio. A specific method for the preparation of these resinous copolymers is described in U.S. Pat. No. 2,676,182, wherein a silica hydrosol is reacted under acidic conditions with a source of triorganosiloxy units such as a hexaorganodisiloxane, for example, hexamethyldisiloxane, or a hydrolyzable triorganosilane, for example, trimethylchlorosilane, or mixtures thereof.

The polysiloxanes (b) having silicon bonded hydrogen may alternatively include the mixtures of polysiloxanes as described above and can also be used herein.

Preferably the polysiloxane (b) having silicon bonded hydrogen atoms has from 0.0001 mol/g to 5 mol/g hydrogen atoms based on the weight of the polymer.

Suitable polysiloxanes (c) terminated with silicon bonded hydrogen include those having units according to the general formula R₂SiO_{1/2} in which each R represents a monovalent hydrocarbon group containing 1 to 20 carbon atoms, such as alkyl (e.g., methyl, ethyl, propyl or butyl) or phenyl groups and p is 0, 1, or 2, and in which the terminal groups have the formula HR₂SiO_{1/2}. It is preferred that each R represents a methyl group.

Preferably the polysiloxane (c) terminated with silicon bonded hydrogen atoms has from 0.0001 mol/g to 2 mol/g hydrogen atoms based on the weight of the polymer.

Suitable polysiloxanes (b) and (c) having silicon bonded hydrogen include those having viscosities on the order of from about 1 mm²/s to about 1000 mm²/s.

Suitable polysiloxanes having silicon bonded unsaturated groups (a) are those with sufficient unsaturated groups for formation of the polymer network. For example, polysiloxanes having siloxane units according to the general formula RmR′SiO(3-m/2) in which each R represents a monovalent hydrocarbon group having 1 to 20 carbon atoms such as alkyls (e.g., methyl, ethyl, propyl or butyl) or phenyl groups, m is 0, 1 or 2 and R′ represents an aliphatically unsaturated group such as vinyl, allyl, hexenyl and cyclohexenyl or a group R″CH═CHR′″, where R″ represents a divalent aliphatic chain linked to the silicon atom and R′″ represents a hydrogen atom or an alkyl group. Preferably, R is methyl.

The polysiloxanes having silicon bonded unsaturated groups can also comprise copolymers having, for instance, units RnSiO(4-n/2) in which R is as referred to above, and n is 0, 1, 2 or 3.

The polysiloxanes having silicon bonded unsaturated groups can also comprise functional siloxane (silicone) resins with unsaturated groups. The functional siloxane resin structure may comprise R₃SiO_{1/2} units (M units) and SiO_{4/2} units (Q units) wherein each R is independently a linear, branched or cyclic hydrocarbon group having 1-20 carbon atoms. R can be unsubstituted or substituted with halogen atoms. Each R can be identical or different, as desired. The hydrocarbon group of R can be exemplified by alkyl groups such as methyl, ethyl, propyl, butyl, hexyl, octyl, vinyl, hexenyl, 3,3,3-trifluoropropyl, chloromethyl, and decyl, cycloaliphatic groups such as cyclohexyl, aryl groups such as phenyl, tolyl, and xylyl, chlorophenyl, and aralkyl groups such as benzyl, styryl and alpha-methylstyryl. The resin can also contain triorganosiloxy units (T units), for example 0.5 to 1 triorganosiloxy group for every SiO_{4/2} unit, alternatively 0.6 to 0.9 triorganosiloxy group for every SiO_{4/2} unit.

The functional siloxane resin structure may comprise RSiO_{3/2} units also known as T units) in Which the groups R, which can be different in different siloxane units, are selected from hydroxyl, hydrocarbon, substituted hydrocarbon, hydrocarbonoxy and substituted hydrocarbonoxy groups. The silicone resin optionally also comprises R₂SiO_{2/2} units (D units) and/or R₃SiO_{1/2} units (M units), in which each R is defined as above. The hydrocarbon and hydrocarbonoxy groups each preferably contain 1 to 20, more preferably 1 to 8, carbon atoms.

Resins comprising R₃SiO_{1/2} units and SiO_{4/2} units are well known in the art. These copolymers are described, for example, in U.S. Pat. Nos. 3,936,582, 2,676,182, and 2,857,356. The resinous copolymers can be prepared by cohydrolysis of a mixture of silanes having four hydrolyzable groups, e.g., silicon tetrachloride, and triorganosilanes having one hydrolyzable group, e.g., trimethylchlorosilane, in the proper ratio. A specific method for the preparation of these resinous copolymers is described in U.S. Pat. No. 2,676,182, wherein a silica hydrosol is reacted under acidic conditions with a source of triorganosiloxy units such as a hexaorganodisiloxane, for example, hexamethyldisiloxane, or a hydrolyzable triorganosilane, for example, trimethylchlorosilane, or mixtures thereof.

Preferably, the polysiloxanes having silicon bonded unsaturated groups have from 0.00001 mol/g as vinyl group based on the weight of the polymer to 2 mol/g as vinyl group based on the weight of the polymer and a viscosity on the order of about 3 mm²/s to about 600,000 mm²/s at 25° C.

Mixtures of polysiloxanes having silicon-bonded unsaturated groups can also be used herein.

The catalysts (d) used in the present invention comprise those known in the art to facilitate the hydrosilylation reaction. These include, for example, platinum and rhodium materials. These catalysts may take any of the known forms such as platinum or rhodium deposited on carriers such as silica gel or powdered charcoal or other appropriate compounds such as platinic chloride, salts of platinum and chloroplatinic acids. A preferred material is chloroplatinic acid either as the commonly obtainable hexahydrate or the anhydrous form because of its easy dispersibility in organosilicon systems and its non-effect on colour of the mixture. Platinum or rhodium complexes may also be used e.g. those prepared from chloroplatinic acid hexahydrate and divinyltetramethyldisiloxane.

When the polysiloxanes and the catalyst of the invention are mixed, they cure at room temperature (20±5° C.) within 10 minutes or, more preferably, within five minutes or less. Higher temperature, such as skin temperature is beneficial as it can decrease the cure time. In order to achieve satisfactory cure it is important that the ratio of silicon-bonded hydrogen atoms of the polysiloxanes to all groups reactive therewith in the formulation (e.g., the unsaturated groups) is appropriate to affect the desired cure. The curing time is dependent on various factors including the type and proportion of other component materials present in the formulation. Working with low or intermediate viscosity materials (<10,000 mm2/s), having a RHV>1.5 and a Pt level between 10 ppm and 200 ppm are factors that allow for that short curing time.

The proportion of cured binder matrix derived from vehicle Y in the controlled release composition may vary widely depending on the intended site of application and the use of the composition. For example, the controlled release compositions may contain from 30% to 99.99% by weight of such cured binder matrix.

The final sustained release composition can be in the form of a gel or an elastomer and it can have pores (e.g., foams) or it can be pore-free.

If it is desired to prolong the cure time, one may include in the formulation one of the known catalyst inhibitors such as cyclic polymethylvinylsiloxane compounds or an acetylenic alcohol e.g. methyl butynol but these are not generally preferred in a formulation according to the invention.

If foaming of the formulation is desired, it may be induced by, for example, including a polysiloxane having silicon-bonded hydroxyl groups which reacts with the polysiloxane having silicon-bonded hydrogen atoms as more fully described, for example, in U.S. Pat. No. 4,026,845.

Alternatively aliphatic alcohol (for example a primary aliphatic or araliphatic alcohol such as a lower aliphatic monofunctional alcohol having up to 12 carbon atoms (e.g. ethanol, n-propanol, or benzyl alcohol), a silanol or a volatile blowing material can be included in the formulation as more fully described, for example, in U.S. Pat. No. 4,550,125.

Preferred foamable formulations include compounds having silicon-bonded or carbon bonded hydroxyl groups which foam and cure in presence of a platinum catalyst according to the patent EP 0 865 787.

The formulation of the invention can be complemented with selected additives to deliver targeted performances without adversely affecting any of the component materials of the formulation or the curing time.

For example the additive can be a compound helping to adjust the rheology behaviour of the formulation or compatibilize the various components of the formulation. The compound can be a liquid material, sometimes called a diluent, or another material, like a gel or a dispersion of solid particles. The compound which may be employed includes volatile and non volatile fluids such as silicone volatiles, silicone fluids, hydrocarbons, alcohols, ketones, esters and any other liquid material. Examples of liquid compounds include hexamethyldisiloxane, octamethyltrisiloxane, and other linear siloxanes, cyclic siloxanes such as octamethylcyclotetrasiloxane, decamethylcyclopentasiloxane and dodecamethylcyclohexasiloxane. Examples also include isododecane, isohexadecane, ethylacetate, ethyl alcohol, isopropyl alcohol, ester palmitate, propylene glycol, C12-15 alkyl benzoate, Caprylic/capric triglyceride, Coco-caprylate/caprate, Diisopropyl adipate, Diisostearyl fumarate, Diisostearyl malate, Isocetyl stearate, Isopropyl isostearate, Isopropyl laurate, Isopropyl myristate, Isopropyl palmitate, Isopropyl stearate, Isostearyl benzoate, Myristyl ether acetate w/propylene glycol, Myristyl lactate, Octyldodecyl stearoyal stearate, Octylpalmitate, Octylstearate, Tridecyl neopentanoate, Triisocetyl citrate, Lauryl alcohol, Oleyl alcohol, Glyceryl trioctanate, Polyglyceryl-3 diisostearate, Mineral oil, Dipropylene glycol, Glycol ether, Glycerin, Castor oil, Lanolin oil, Sunflower oil, Isododecane, C11-12 isoparaffin, Polydecene.

The additive can be a gel elastomeric silicone like the so-called elastomer blend, silicone polyethers, film formers like silicone acrylate dispersion or silicone polyamide compounds.

For example the additive can be water. The water can be either emulsified in components of the vehicle Y or the components of the vehicle Y can be emulsified in the water phase.

For example the additive can be a surfactant or an emulsifier to compatibilize the various components of the formulation. The surfactant or the emulsifier which may be employed includes silicone polyethers.

For example the additive can be a filler to adjust the rheology behaviour or the physical properties or to compatibilize the various components of the formulation. The term filler comprises any solid material. The fillers which may be employed include, but are not limited to siloxane resin, rosin type resins, acrylic polymer resins, polysaccharides, carbomer, alginate, zinc oxide, ground, precipitated, and colloidal calcium carbonates which can be untreated or treated with stearate or stearic acid; reinforcing silicas such as fumed silicas, precipitated silicas, and hydrophobed silicas; crushed quartz, ground quartz, alumina, aluminium hydroxide, titanium dioxide, diatomaceous earth, iron oxide, carbon black, and graphite.

For example, the filler can be silica to provide the following benefits when used in the following ranges: 0.1 wt. % to 5 wt. % compatibilizer and drying agent/5 wt. % to 15 wt. % rheological modifier and texture enhancer/15 wt. % to 30 wt. % mechanical strength enhancer.

For example the additive can be a cosmetic excipient or a pharmaceutical excipient to provide complementary benefits. The complementary benefits which may be delivered includes emolliency, partial or complete occlusivity, sensory benefits, colour. The cosmetic excipient or the pharmaceutical excipient which may be employed includes colorants, coloured indicators, other diluents, excipients employed in pharmacy, compounds intended to perform as pH buffers in controlling the environment immediately in and around the formulation, stabilizers, preservatives, surfactants for cellular formulations such as fluorinated silicones, absorbents for wounds, alginate, polysaccharides, gelatin, collagen, and materials that can decrease the friction on the surface of the cured film and/or change its gloss.

Some additional examples of the cosmetics, personal care, and cosmeceutical ingredients and pharmaceutical excipients that may be used herein may be found in the CTFA ingredient Database and the handbook of pharmaceutical excipients and can include, for example, absorbents, anticaking agents, antioxidants, antistatic agents, astringents, binders, buffering agents, bulking agents, chelating agents, colorants, cosmetic astringents, cosmetic biocides, deodorant agents, emollients, external analgesics, film formers, flavouring agents, fragrance ingredients, humectants, lytic agents, moisturizing agents, occlusivity enhancers. opacifying agents, oxidizing and reducing agents, penetration enhancers, pesticides, plasticizers, preservatives, skin bleaching agents, skin conditioning agents, skin protectants, slip modifiers, solubilising agents, solvents, sunscreen agents, surface modifiers, surfactants and emulsifying agents, suspending agents, thickening agents, viscosity controlling agents including increasing or decreasing agents, UV light absorbers.

Cosmetic, personal care and cosmeceutical ingredients, and pharmaceutical excipients which may be employed are selected, for example, from the following chemical classes: alcohols, fatty alcohols and polyols, aldehydes, alkanolamines, alkoxylated alcohols (e.g. polyethylene glygol derivatives of alcohols and fatty alcohols), alkoxylated amides, alkoxylated amines, alkoxylated carboxylic acids, amides including salts (e.g. ceramides), arnines, amino acids including salts and alkyl substituted derivatives, esters, alkyl substituted and acyl derivatives, polyacrylic acids, acrylamide copolymers, adipic acid copolymers, alcohols, aminosilicones, biological polymers and derivatives, butylene copolymers, carbohydrates (e.g. polysaccharides, chitosan and derivatives), carboxylic acids, carbomers, esters, ethers and polymeric ethers (e.g. PEG derivatives, PPG derivatives), glyceryl esters and derivatives, halogen compounds, heterocyclic compounds including salts, hydrophilic colloids and derivatives including salts and gums (e.g. cellulose derivatives, gelatin, xanthan gum, natural gums), imidazolines, inorganic materials (clay, Ti02, ZnO), ketones (e.g. camphor), isethionates, lanolin and derivatives, organic salts, phenols including salts (e.g. parabens), phosphorus compounds (e.g. phosphate derivatives), polyacrylates and acrylate copolymers, protein and enzymes derivatives (e.g. collagen), synthetic polymers including salts, siloxanes and silanes, sorbitan derivatives, sterols, sulfonic acids and derivatives and waxes.

Some examples of anti acne agents are salicylic acid and sulfur. Some examples of antifungal agents are calcium undecylenate, undecylenic acid, zinc undecylenate, and povidone-iodine.

Some examples of antimicrobial agents are alcohol, benzalkonium chloride, benzethonium chloride, hydrogen peroxide, methylbenzethonium chloride, phenol, poloxamer 188, and povidone-iodine. Some examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, camellia sinensis oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadienelt-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodiurn rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, Isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, Nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (melaleuca aftemifolia) oil, tocopheryl acetate, tetrahexyldecyl ascorbate, tetrahydrodiferuloylmethane, tocopheryl linoleateioleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl)phosphite, ubiquinone, and zinc dibutyldithiocarbarnate. Some examples of cosmetic biocides are aluminium phenolsulfonate, ammonium phenolsulfonate, bakuchiol, benzalkonium bromide, benzalkonium cetyl phosphate, benzalkonium chloride, benzalkonium saccharinate, benzethonium chloride, potassium phenoxide, benzoxiquine, benzoxonium chloride, bispyrithione, boric acid, bromochlorophene, camphor benzalkonium methosulfate, captan, cetalkoniurn chloride, cetearalkonium bromide, cetethyldimonium bromide, cetrimonium bromide, cetrimonium chloride, cetrimonium methosulfate, cetrimonium saccharinate, cetrimonium tosylate, cetylpyridinium chloride, chloramine t, chlorhexidine, chlorhexidine diacetate, chlorhexidine digluconate, chlorhexidine dihydrochloride, p-chloro-m-cresol, chlorophene, p-chlorophenol, chlorothyrnol, chloroxylenol, chlorphenesin, ciclopirox olamine, climbazole, cloflucarban, clotrimazole, coal tar, colloidal sulfur, o-cytnen-5-01, dequalinium acetate, dequalinium chloride, dibromopropamidine diisethionate, dichlorobenzyl alcohol, dichlorophene, dichlorophenyl imidazoldioxolan, dichloro-m-xylenol, diiodomethyltolylsulfone, dimethylol ethylene thiourea, diphenylmethyl piperazinylbenzimidazole, domiphen bromide, 7-ethylbicyclooxazolidine, fluorosalan, formaldehyde, glutaral, hexachlorophene, hexamidine, hexamidine diisethionate, hexamidine diparaben, hexamidine paraben, hexetidine, hydrogen peroxide, hydroxymethyl dioxoazabicyclooctane, ichthammol, isopropyl cresol, lapyrium chloride, lauralkonium bromide, lauralkonium chloride, laurtrimonium bromide, laurtrimonium chloride, laurtrimonium trichlorophenoxide, lauryl isoquinolinium bromide, lauryl isoquinolinium saccharinate, laurylpyridinium chloride, mercuric oxide, methenamine, methenammonium chloride, methylbenzethonium chloride, myristalkonium chloride, myristalkonium saccharinate, myrtrimonium bromide, nonoxynol-9 iodine, nonoxynol-12 iodine, olealkonium chloride, oxyquinoline, oxyquinoline benzoate, oxyquinoline sulfate, PEG-2 coco-benzonium chloride, PEG-10 coco-benzonium chloride, PEG-6 undecylenate, PEG-8 undecylenate, phenol, o-phenylphenol, phenyl salicylate, piroctone olamine, sulfosuccinylundecylenate, potassium o-phenylphenate, potassium salicylate, potassium troclosene, propionic acid, pvp-iodine, quaternium-8, quaternium-14, quaternium-24, sodium phenolsulfonate, sodium phenoxide, sodium o-phenylphenate, sodium shale oil sulfonate, sodium usnate, thiabendazole, 2,2′-thiobis(4-chlorophenol), thiram, triacetin, triclocarban, triclosan, trioctyldodecyl borate, undecylenamidopropylamine oxide, undecyleneth-6, undecylenic acid, zinc acetate, zinc aspartate, zinc borate, zinc chloride, zinc citrate, zinc cysteinate, zinc dibutyldithiocarbamate, zinc gluconate, zinc glutamate, zinc lactate, zinc phenolsulfonate, zinc pyrithione, zinc sulfate, and zinc undecylenate. Some examples of external analgesics are benzyl alcohol, capsicum oleoresin (capsicum Frutescens oleoresin), methyl salicylate, camphor, phenol, capsaicin, juniper tar (juniperus oxycedrus tar), phenolate sodium (sodium phenoxide), capsicum (capsicum frutescens), menthol, resorcinol, methyl nicotinate, and turpentine oil (turpentine). Some examples of oxidizing agents are ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, and zinc peroxide. Some examples of reducing agents are ammonium bisulfite, ammonium sulfite, ammonium thioglycolate, ammonium thiolactate, cystemaine hci, cystein, cysteine HCI, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, and zinc formaldehyde sulfoxylate. An example of a skin bleaching agent is hydroquinone. Some examples of skin protectants are allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, and zinc oxide. Some examples of sunscreen agents are aminobenzoic acid, cinoxate, diethanolamine methoxycinnamate, digalloyl trioleate, dioxybenzone, ethyl 4-[bis(hydroxypropyl)]aminobenzoate, glyceryl aminobenzoate, homosalate, lawsone with dihydroxyacetone, menthyl anthranilate, octocrylene, octyl methoxycinnarnate, octyl salicylate, oxybenzone, padimate 0, phenylbenzimidazole sulfonic acid, red petrolatum, sulisobenzone, titanium dioxide, and trolamine salicylate. Some examples of UV light absorbing agents are acetaminosalol, allatoin PABA, benzalphthalide, benzophenone, benzophenone 1-12,3-benzylidene camphor, benzylidenecamphor hydrolyzed collagen sulfonamide, benzylidene camphor sulfonic acid, benzyl salicylate, bornelone, bumetriozole, butyl methoxydibenzoylmethane, butyl PABA, ceridsilica, ceridsilica talc, cinoxate, dea-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl p-methoxycinnamate, isopropylbenzyl salicylate, isopropyl dibenzolylmethane, isopropyl methoxycinnamate, menthyl anthranilate, menthyl salicylate, 6methylbenzylidene, camphor, octocrylene, octrizole, octyl dimethyl PABA, octyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, tea-phenylbenzimidazole sulfonate, tea-salicylate, terephthalylidene dicamphor sulfonic acid, titanium dioxide, tripaba panthenol, urocanic acid, and va-crotonates/methacryloxybenzophenone-1 copolymer.

For example the additive can be a hydrophilic material that can provide bioadhesive, modulating water absorption, swellability or controlled release properties as set forth in EP 465,744. Such additives include, but are not limited to carbomer (polyacrylic acid), polysaccharides, sugars and derivatives, polyvinyl alcohol, glycerin, polyether glycols.

Since mixing of the component materials in vehicle Y causes curing at room temperature, these component materials can be stored in a plurality of containers prior to use to inhibit curing. For instance, one container could contain the catalyst and a second could contain the polysiloxanes. Alternatively, the catalyst could be mixed with one of the siloxanes in one container and the other siloxane could be in a second container. Each of the additional components in the formulation is put in the container which is most desirable depending on factors such as stability, viscosity, and interactions.

Another alternative to prevent the vehicle Y to cure prior its use comprises temporarily inhibiting the hydrosilylation catalyst (d) by encapsulating the hydrosilylation catalyst (d) or by adding transient inhibitors.

According to the method of the invention, the spreadable formulation comprising the vehicle Y, the active agent X and any other optional components are delivered and spread onto the desired site in a manner which causes mixing of the component materials. The formulation cures after being applied and results in a sustained release composition. Preferably, the spreadable formulations are spread onto a biological surface including, but not limited to animal bodies (e.g., human or other animal).

The delivery herein is performed by conventional techniques known in the art. For instance the delivery systems include, but not limited to can, tube, sachet, syringe, stick, pencil, brush, sponge, wet stamp and roll-on as known in the art. These delivery devices can comprise one or more than one chamber according to the need to separate the components of the formulation.

Whichever of the above means of delivery is chosen, the formulation components are delivered and spread to the desired site. Mixing of the formulation components can occur either in the delivery packaging, during the delivery or during the spreading onto the desired site.

For instance, a mixing chamber can be built into the delivery packaging such that as the formulation components are drawn or forced out of their separate containers they are mixed prior to being delivered.

In an embodiment the formulation components are separated by fragile walls which can easily be broken to allow the formulation components for getting in contact together. The mixing then occurs by hand kneading or with a mixing tool as known in the art.

In another embodiment the formulation components are forced into a mixing device such as a static mixer and then delivered to the site.

In still another embodiment, the formulation components can be delivered sequentially and then be mixed on the desired site.

In case of one part system in which the catalyst is encapsulated or temporarily inhibited, an external factor triggers the cure by releasing the catalyst. For instance, such factors can be, but are not limited to elevated temperature (e.g. body or skin temperature, hair dryer), shearing effect or evaporation of certain formulation additives (e.g. diluent).

The present invention offers numerous advantages over the prior art. The method described herein allows for the simple dispensing of the sustained release composition to various sites of application including the face and the areas which cannot be treated with conventional adhesive patches or sprayed compositions. As such, a skilled practitioner is not required for application. Likewise, the formulation chosen enables sustained release compositions to be formed by simple and easily sustained methods in situ. Moreover, the sustained release composition can be formed into a wide variety of shapes and have selected combinations of properties (e.g. bioadhesion, release rate and release profile). Preferably, the invention provides silicone containing compositions able to form adhesive films on substrates, which typically comprises a curable silicone formulation containing:
(a) a polyorganosiloxane polymer having at least two functional SiVi groups per molecule, each SiVi group containing an alkenyl functionality directly bonded to a silicon atom,
(b) a crosslinker polyorganosiloxane compound having at least 3 Si-bonded hydrogen groups or SiH groups per molecule,
(c) a chain extender compound which is a telechelic polyorganosiloxane having terminal SiH groups,
(d) a hydrosilation catalyst for the reaction of SiH groups with SiVi groups, with RHV>1.5 wherein RHV is the ratio of the number of SiH moles in (b) and (c) to the number of SiVi moles in (a) and (d), and 0<RHC<0.7 wherein RHC is the ratio of the number of SiH moles in (c) to the number of SiH moles in (b) and (c).

Similarly, the formulations and the sustained release compositions described herein can easily comprise the attributes of cosmetic products in term of ease of use and aesthetic and still deliver the benefits of pharmaceutical therapeutic delivery systems with active sustained release property.

The formulation and sustained release composition herein are generally acceptable on many biological membranes. The sustained release composition may be formed on intact or damaged skin or in a natural or artificial cavity of the body. The cavity may be, for example, the ocular, oral (mouth), nasal, aural, vaginal or rectal cavity or a cavity formed, for example, in a tooth or an open wound.

The compositions may be formulated to give a moderate to rapid release of active agent X. The drug delivery profile of compositions according to the invention may be predetermined by appropriate selection of the types and proportions of component materials and ingredients used.

It is a further advantage of the present invention that the controlled release compositions can have many physical properties from gel to elastomer and foam so that they are able to withstand many of the pressures exerted during normal activities of a patient.

### Examples

To clarify the invention, Examples follow which illustrate the methods of the invention. Unless indicated, all parts are by weight and all viscosities are at 25° C.

The actives Y used to illustrate the invention are:

| Name | Active Molecules | Therapeutic Indications |
|---|---|---|
| Centella | Saponins | Wound healing and |
| asiatica | | treatment of skin (varicose |
| (Gotu Kola) | | ulcers, eczema, and |
| | | psoriasis) |
| Arnica | Sesquiterpene lactone | Topical remedy for injuries |
| Montana | | such as sprains and bruises. |
| Eucalyptol | 1,3,3-trimethyl- | Inflammation and pain |
| | 2-oxabicyclo[2,2,2]octane | relief, cough |
| | | suppressant |
| Calcium | Polysaccharide of | Haemostatics, wound |
| alginate | mannuronic and glucuronic | healing |
| | acids | |
| Menthol | 2-(2-Propyl)-5- | Analgesic, decongestant |
| | methylcyclohexanol | and cooling effect |
| Green tea | Epigallocathechin gallate | Potent antioxidant, free |
| | | radical scavenger, enzyme |
| | | inhibition or activation |
| Acetaminophen | 4-acetylaminophenol | Analgesic, antipyretic |
| (Paracetamol) | | |
| Benzocain | 4-Aminobenzoic acid ethyl | Local anaesthetic |
| | ester | |

The additives used to illustrate the invention are:

| Name | Benefits |
|---|---|
| Zinc oxide | UV screen agent, skin care |
| Propylene glycol | Solvent, hydrophilic agent |
| Decamethylpentacyclosiloxane | Solvent |
| Silica (in situ treated) | Reinforcement |

The formulation components X used to illustrate the invention are:

| Formulation | | Viscosity | vinyl | H as SiH |
|---|---|---|---|---|
| component | Description | (mm2/s) | (mol/g) | (mol/g) |
| (a) 1 | Vinyl-terminated | 9,000 | 5E−5 | NA* |
| | polydimethylsiloxane | | | |
| (a) 2 | Vinyl-terminated | 2,000 | 8.5E−5 | NA |
| | polydimethylsiloxane | | | |
| (a) 3 | Vinyl-terminated | 400 | 1.7E−4 | NA |
| | polydimethylsiloxane | | | |
| (a) 4 | Vinyl-terminated | 60,000 | 3.2E−5 | NA |
| | polydimethylsiloxane | | | |
| (b) | Dimethyl, | 5 | NA | 7.8E−3 |
| | methylhydrogen | | | |
| | siloxane | | | |
| (c) | Dimethylhydrogen- | 10 | NA | 1.8E−3 |
| | terminated | | | |
| | polydimethylsiloxane | | | |
| (d) | Platinum complex | 400 | 8.4E−4 | NA |

*NA = not applicable
1.8E−3 = 1.8.10⁻³

The formulations used to illustrate the invention are:

| Formulation component | F1 | F2 | F3 | F4 |
|---|---|---|---|---|
| (a) 1 | 94.10 wt. % | 95.50 wt. % | 92.42 wt. % | 96.74 wt. % |
| (b) | 1.20 wt. % | 1.65 wt. % | 0.64 wt. % | 2.06 wt. % |
| (c) | 3.50 wt. % | 1.65 wt. % | 5.78 wt. % | 0.00 wt. % |
| (d) | 1.20 wt. % | 1.20 wt. % | 1.16 wt. % | 1.20 wt. % |
| RHV | 2.74 | 2.74 | 2.75 | 2.75 |
| RHC | 0.40 | 0.19 | 0.68 | 0.00 |

Procedure to prepare the evaluation samples of the formulations above and their versions loaded with actives and/or additives:
1) Disperse the active or the additive in the solvent if necessary
2) Weigh (a)
3) Weigh (b) and add to (a) and mix at 800 rpm
4) Weigh (c) and add to the blend 3) and mix at 800 rpm
5) Add the active or the additive to the blend 4) and mix at 800 rpm
6) Add the catalyst (d) to the blend 5) and mix at 800 rpm
7) Coat with a blade the blend 6) onto a polyester film (Mylar®) to obtain a thickness of 100 microns.
8) Let cure at room temperature RT (23° C.+/−2° C.)
9) Evaluate the cure time (or snap time) by gently touching the film with a clean finger:

the film is said cured when no transfer of material is observed on the finger.
10) Evaluate visually the film formed with the following scale: 1, 3 and 5 with 1=low, 3=medium and 5=high for the following criteria:
   Adhesion as adhesion to polyester substrate (Mylar®)—evaluate how difficult it is to peel of a piece of the cured film from the Mylar.
   Tack as level of adhesion to clean finger.
   Cohesiveness as the capacity of the cured film to be peeled off from the Mylar in large pieces (about 1 cm×1 cm piece).
   Conformability as the plastic elongation of the cured film tested by stretching the cured film.

The formulations loaded with actives are F1/x, F2/x, F3/x and F4/x, with x=8 to 21

### Example 1

| Formulation | | | | |
|---|---|---|---|---|
| component | F1 | F2 | F3 | F4 |
| RHV | 2.74 | 2.74 | 2.75 | 2.75 |
| RHC | 0.40 | 0.19 | 0.68 | 0.00 |
| Cure time at RT | <6 | <6 | <6 | <6 |
| (min) | | | | |
| Softness (mm) | 31.5 | Not done | 38 | 12.3 |
| with 62.5 g | | | | |
| penetration probe | | | | |
| Adhesion to | 3 | 3 | 5 | 1 |
| Mylar | | | | |
| Tack | 1 | 1 | 5 | 1 |
| Cohesiveness | 3 | 3 | 1 | 5 |
| Conformability | 5 | 5 | 1 | 3 |

The formulation with no chain extender (F4, RHC=0) leads to a film that is considered not tacky but an insufficient adhesion level. Formulation with the highest level of chain extender (F3, RHC=0.67-0.68) leads to a film with a high adhesion level but which is too tacky, although this tackiness could be decreased by adding some powder filler in the formulation Formulations F1 (RHC=0.40) & F2 (RHC=0.19) have been shown as being the best compromise between adhesion and tack.

### Example 2

### Addition of Silica

| Formulation | | | | | | | |
|---|---|---|---|---|---|---|---|
| component | S1 | S2 | S3 | S4 | S5 | S6 | S7 |
| (a) 2 | 94.10 wt. % | 91.28 wt. % | 89.39 wt. % | 84.69 wt. % | 79.98 wt. % | 75.28 wt. % | 70.57 wt. % |
| Silica | 0 | 3 wt. % | 5 wt. % | 10 wt. % | 15 wt. % | 20 wt. % | 25 wt. % |
| (b) | 1.20 wt. % | 1.16 wt. % | 1.14 wt. % | 1.08 wt. % | 1.02 wt. % | 0.96 wt. % | 0.90 wt. % |
| (c) | 3.50 wt. % | 3.40 wt. % | 3.33 wt. % | 3.15 wt. % | 2.98 wt. % | 2.80 wt. % | 2.63 wt. % |
| (d) | 1.20 wt. % | 1.16 wt. % | 1.14 wt. % | 1.08 wt. % | 1.02 wt. % | 0.96 wt. % | 0.90 wt. % |
| RHV | 1.74 | 1.74 | 1.74 | 1.74 | 1.74 | 1.74 | 1.74 |
| RHC | 0.40 | 0.40 | 0.40 | 0.40 | 0.40 | 0.40 | 0.40 |
| Cure time at | <6 | <6 | <6 | <6 | <6 | <6 | <6 |
| RT (min) | | | | | | | |

When used, silica SiO2 is added to the vinyl polymer with mixing and then treated in-situ using a trimethylsilyl-capping agent that is added to the blend.

The formulation of the invention can be complemented with silica without impacting the cure time.

### Example 3

### Addition of Platinum Complex Catalyst

| Formulation | | | | | | |
|---|---|---|---|---|---|---|
| component | Pt 2 | Pt 3 | Pt 4 | Pt 5 | Pt 1 | Pt 6 |
| (a)1 | 95.18 | 95.06 | 94.82 | 94.34 | 94.10 | 93.80 |
| (b) | 1.20 | 1.20 | 1.20 | 1.20 | 1.20 | 1.20 |
| (c) | 3.50 | 3.50 | 3.50 | 3.50 | 3.50 | 3.50 |
| (d) | 0.12 | 0.24 | 0.48 | 0.96 | 1.20 | 1.50 |
| RHV | 3.22 | 3.16 | 3.04 | 2.83 | 2.74 | 2.63 |
| RHC | 0.40 | 0.40 | 0.40 | 0.40 | 0.40 | 0.40 |
| Cure time at RT (min) | 9.5 | 8.5 | 8 | 6.5 | 6 | <1 |

The addition of platinum complex catalyst decreases the cure time.

### Example 4

### Formulation with Vinyl-Terminated Polydimethylsiloxanes Having Different Molecular Weights

With keeping the recipes identical:

| Formulation | | | | |
|---|---|---|---|---|
| component | V1 | V2 | V3 | V4 |
| (a)1 | 94.10 | | | |
| (a)2 | | 94.10 | | |
| (a)3 | | | 94.10 | |
| (a)4 | | | | 94.10 |
| (b) | 1.20 | 1.20 | 1.20 | 1.20 |
| (c) | 3.50 | 3.50 | 3.50 | 3.50 |
| (d) | 1.20 | 1.20 | 1.20 | 1.20 |
| RHV | 2.74 | 1.00 | 0.94 | 3.91 |
| RHC | 0.40 | 0.40 | 0.40 | 0.40 |
| Cure time at RT (min) | 7.5 | 8 | 8 | <1 |

With keeping the RHVs similar, around 3:

| Formulation | | | | |
|---|---|---|---|---|
| component | V5 | V6 | V7 | V8 |
| (a)1 | 93.40 | | | |
| (a)2 | | 90.70 | | |
| (a)3 | | | 84.70 | |
| (a)4 | | | | 94.90 |
| (b) | 1.40 | 2.10 | 3.60 | 1.00 |
| (c) | 4.00 | 6.00 | 10.50 | 2.90 |
| (d) | 1.20 | 1.20 | 1.20 | 1.20 |
| RHV | 3.19 | 3.11 | 3.11 | 3.23 |
| RHC | 0.40 | 0.40 | 0.40 | 0.40 |
| Cure time at RT (min) | 5 | 4.5 | 3 | <1 |

The formulations of the invention can be obtained from different vinyl-terminated polydimethylsiloxanes.

### Example 5

### Variation of RHV from 4 to 10

| Formulation | | | | | |
|---|---|---|---|---|---|
| component | V9 | V10 | V11 | V12 | V13 |
| (a)1 | 91.90 | 90.00 | 88.00 | 86.00 | 82.60 |
| (b) | 1.80 | 2.30 | 2.80 | 3.30 | 4.20 |
| (c) | 5.10 | 6.60 | 8.00 | 9.50 | 12.00 |
| (d) | 1.20 | 1.20 | 1.20 | 1.20 | 1.20 |
| RHV | 4.14 | 5.41 | 6.70 | 8.07 | 10.58 |
| RHC | 0.40 | 0.40 | 0.40 | 0.40 | 0.40 |
| Cure time at | 3.5 | 2 | <1 | <1 | <1 |
| RT (min) | | | | | |

Increasing the RHV allows for decreasing the cure time.

### Example 6

### Use of Blends of Different Vinyl-Terminated Polydimethylsiloxanes

| Formulation | | | | |
|---|---|---|---|---|
| component | WW1 | WW2 | WW3 | WW4 |
| (a)1 | 47.10 | 71.10 | 24.10 | 44.00 |
| (a)3 | 47.00 | 23.00 | 70.00 | 43.80 |
| (b) | 1.20 | 1.20 | 1.20 | 2.80 |
| (c) | 3.50 | 3.50 | 3.50 | 8.20 |
| (d) | 1.20 | 1.20 | 1.20 | 1.20 |
| RHV | 1.40 | 1.86 | 1.13 | 3.48 |
| RHC | 0.40 | 0.40 | 0.40 | 0.40 |
| Cure time at RT (min) | 7.5 | 7.5 | <7 | 3.5 |
| (a)1 | 47.10 | 71.10 | 24.10 | 44.00 |
| (a)2 | 47.00 | 23.00 | 70.00 | 43.80 |
| (b) | 1.20 | 1.20 | 1.20 | 2.80 |
| (c) | 3.50 | 3.50 | 3.50 | 8.20 |
| (d) | 1.20 | 1.20 | 1.20 | 1.20 |
| RHV | 2.13 | 2.40 | 1.92 | 5.27 |
| RHC | 0.40 | 0.40 | 0.40 | 0.40 |
| Cure time at RT (min) | 4.5 | 4 | 7 | <2 |

The formulations of the invention can be obtained from combination of different vinyl-terminated polydimethylsiloxanes.

### Example 7

### Variation of the RHC and Impact on Cure Time

| Formulation | | | | | | | |
|---|---|---|---|---|---|---|---|
| component | H1 | H2 | H3 | H4 | H5 | H6 | H7 |
| (a)1 | 91.60 | 92.10 | 93.00 | 93.70 | 94.50 | 95.10 | 95.80 |
| (b) | 0.80 | 0.90 | 1.10 | 1.30 | 1.50 | 1.80 | 2.00 |
| (c) | 6.40 | 5.80 | 4.70 | 3.80 | 2.80 | 1.90 | 1.00 |
| (d) | 1.20 | 1.20 | 1.20 | 1.20 | 1.20 | 1.20 | 1.20 |
| RHV | 3.18 | 3.11 | 3.01 | 2.98 | 2.92 | 3.03 | 3 |
| RHC | 0.65 | 0.60 | 0.50 | 0.40 | 0.30 | 0.20 | 0.10 |
| Cure time at RT | <3.5 | 3.5 | 2.5 | 4.5 | 3 | 3.5 | 5.5 |
| (min) | | | | | | | |

### Example 8

### With 10 wt. % of Propylene Glycol

| Formulation | | | | |
|---|---|---|---|---|
| component | F1/8 | F2/8 | F3/8 | F4/8 |
| Cure time at RT | <6 | <10 | <6 | <6 |
| (min) | | | | |
| Adhesion to | 1 | 3 | 3 | 1 |
| Mylar | | | | |
| Tack | 1 | 1 | 3 | 1 |
| Film | 5 | 5 | 1 | 5 |
| Conformability | 5 | 5 | 1 | 5 |

### Example 9

### With 20 wt. % of Propylene Glycol

| Formulation | | | | |
|---|---|---|---|---|
| component | F1/9 | F2/9 | F3/9 | F4/9 |
| Cure time at RT | <6 | <10 | <6 | <6 |
| (min) | | | | |
| Adhesion to | 1 | 3 | 1 | 1 |
| Mylar | | | | |
| Tack | 1 | 1 | 1 | 1 |
| Film | 5 | 5 | 3 | 5 |
| Conformability | 5 | 5 | 3 | 5 |

### Example 10

### With 1 wt. % Centella Asiatica and 9 wt. % of Propylene Glycol

| Formulation | | | | |
|---|---|---|---|---|
| component | F1/10 | F2/10 | F3/10 | F4/10 |
| Cure time at RT | <6 | <10 | <6 | <6 |
| (min) | | | | |
| Adhesion to | 1 | 3 | 3 | 1 |
| Mylar | | | | |
| Tack | 1 | 1 | 3 | 1 |
| Film | 5 | 5 | 1 | 5 |
| Conformability | 5 | 5 | 1 | 5 |

### Example 11

### With 5 wt. % Centella Asiatica and 15 wt. % of Propylene Glycol

| Formulation | | | | |
|---|---|---|---|---|
| component | F1/11 | F2/11 | F3/11 | F4/11 |
| Cure time at RT | <6 | <10 | <6 | <6 |
| (min) | | | | |
| Adhesion to | 1 | 1 | 1 | NA |
| Mylar | | | | |
| Tack | 1 | 1 | 3 | NA |
| Film | 5 | 5 | 5 | NA |
| Conformability | 5 | 5 | 5 | NA |

### Example 12

### With 1 wt. %, 5 wt. % or 10 wt. % Arnica Tincture

| Formulation | | | | |
|---|---|---|---|---|
| component | F1/12 | F2/12 | F3/12 | F4/12 |
| Cure time at RT | <6 | <6 | <6 | <1 |
| (min) | | | | |
| Adhesion to | 1 | 1 | 3 | 1 |
| Mylar | | | | |
| Tack | 1 | 1 | 3 | 1 |
| Film | 5 | 5 | 3 | 5 |
| Conformability | 5 | 5 | 3 | 5 |

### Example 13

### With 1 wt. %. 5 wt. % or 10 wt. % Calcium Alginate

| Formulation | | | | |
|---|---|---|---|---|
| component | F1/13 | F2/13 | F3/13 | F4/13 |
| Cure time at RT | <6 | <6 | <6 | <6 |
| (min) | | | | |
| Adhesion to | 3 | 3 | 5 | 1 |
| Mylar | | | | |
| Tack | 1 | 1 | 5 | 1 |
| Film | 3 | 3 | 1 | 5 |
| Conformability | 5 | 5 | 1 | 3 |

### Example 14

### With 1 wt. %. 5 wt. % or 10 wt. % Eucalyptol

| Formulation | | | |
|---|---|---|---|
| component | F1/14 | F2/14 | F3/14 |
| Cure time at RT | <6 | <6 | <6 |
| (min) | | | |
| Adhesion to Mylar | 3 | 1 | 5 |
| Tack | 1 | 1 | 3 |
| Film | 3 | 5 | 1 |
| Conformability | 5 | 5 | 1 |

### Example 15

### With 10 wt. % Decamethylpentacyclosiloxane

| Formulation component | F1/15 | F3/15 |
|---|---|---|
| Cure time at RT (min) | <6 | <6 |
| Adhesion to Mylar | 3 | 5 |
| Tack | 1 | 1 |
| Film | 3 | 1 |
| Conformability | 5 | 1 |

### Example 16

### With 1 wt. % menthol and 9 wt. % Decamethylpentacyclosiloxane

| Formulation component | F1/16 | F3/16 |
|---|---|---|
| Cure time at RT (min) | <6 | <6 |
| Adhesion to Mylar | 3 | 5 |
| Tack | 1 | 3 |
| Film | 3 | 1 |
| Conformability | 5 | 1 |

### Example 17

### With 5 wt. % Menthol and 5 wt. % Decamethylpentacyclosiloxane

| Formulation component | F1/17 | F3/17 |
|---|---|---|
| Cure time at RT (min) | <6 | <6 |
| Adhesion to Mylar | 3 | 5 |
| Tack | 3 | 3 |
| Film | 3 | 1 |
| Conformability | 5 | 1 |

### Example 18

### With 1 wt. % Green Tea and 9 wt. % Propylene Glycol

| Formulation component | F3/18 | F4/18 |
|---|---|---|
| Cure time at RT (min) | <6 | <6 |
| Adhesion to Mylar | 5 | 1 |
| Tack | 1 | 1 |
| Film | 3 | 5 |
| Conformability | 5 | 3 |

### Example 19

### With 1 wt. % Acetaminophen and 9 wt. % Propylene Glycol

| Formulation component | F3/19 | F4/19 |
|---|---|---|
| Cure time at RT (min) | <6 | <6 |
| Adhesion to Mylar | 5 | 1 |
| Tack | 1 | 1 |
| Film | 3 | 5 |
| Conformability | 5 | 3 |

### Example 20

### With 1 wt. % Benzocain and 9 wt. % Propylene Glycol

| Formulation component | F3/20 | F4/20 |
|---|---|---|
| Cure time at RT (min) | <6 | <6 |
| Adhesion to Mylar | 5 | 1 |
| Tack | 1 | 1 |
| Film | 3 | 5 |
| Conformability | 5 | 3 |

### Example 21

### With 1 wt. % or 5 wt. % Zinc Oxide

| Formulation component | F3/21 | F4/21 |
|---|---|---|
| Cure time at RT (min) | <6 | <6 |
| Adhesion to Mylar | 5 | 1 |
| Tack | 5 | 1 |
| Film | 1 | 5 |
| Conformability | 1 | 5 |

The formulations of the invention can be loaded with various pharmaceutical excipients and actives such as propylene glycol, Centella asiatica, Arnica tincture, calcium alginate, eucalyptol, decamethylpentacyclosiloxane, menthol, green tea, acetaminophen, benzocain and zinc oxide.

1. (canceled)

2. (canceled)

3. (canceled)

4. (canceled)

5. (canceled)

6. A controlled release composition for medical or pharmaceutical use comprising a spreadable formulation containing an active agent X and a vehicle Y, said vehicle comprising a curable silicone formulation containing:
(a) a polydiorganosiloxane having at least two silicon-bonded alkenyl groups per molecule,
(b) a hydrosilicon compound having at least 3 silicon-bonded hydrogen atoms per molecule,
(c) a diorganohydrogensiloxy-terminated polydiorganosiloxane,
(d) a hydrosilylation catalyst for the reaction of SiH groups with Si-Alkenyl groups, characterised in that the formulation of vehicle Y is such that:
RHV 1.5 preferably RHV>2.5 more preferably RHV>3 wherein RHV is the ratio of the number of SiH moles in (b) and (c) with respect to the number of Si-Alkenyl moles in (a) and (d), and
0<RHC<0.7 wherein RHC is the ratio of the number of SiH moles in (c) with respect to the number of SiH moles in (b) and (c).

7. A composition as claimed in claim 6 wherein the remaining silicon-bonded organic groups of the polydiorganosiloxane (a) are selected from alkyl and aryl groups, said polydiorganosiloxane having a viscosity at 25° C. 50 of from 3 mm²/s to 100,000 mm²/s.

8. A composition as claimed in claim 6, wherein the hydrosilicon compound (b) consists essentially of RHSiO-groups, R2ZSiO-groups and optionally R2SiO-groups and has a viscosity at 25° C. of no more than 1000 mm²/s, wherein R denotes an alkyl or aryl group having no more than 8 carbon atoms, and Z denotes H or R.

9. A composition as claimed in claim 6, wherein the hydrosilicon compound (b) is a linear hydrosilicon compound.

10. A composition as claimed in claim 6, wherein the organic substituents of the diorganohydrogensiloxy-terminated polydiorganosiloxane (c) are alkyl or aryl groups having no more than 8 carbon atoms.

11. A method of making a controlled release composition for medical or pharmaceutical use comprising preparing a spreadable formulation containing an active agent X and a vehicle Y, said vehicle comprising a curable silicone formulation containing:
a. a polydiorganosiloxane having at least two silicon-bonded alkenyl groups per molecule, the remaining silicon-bonded organic groups being selected from alkyl and aryl groups, said polydiorganosiloxane having a viscosity at 25° C. 50 of from 3 mm²/s to 100,000 mm²/s,
b. a preferably linear hydrosilicon compound having at least 3 silicon-bonded hydrogen atoms per molecule and consisting essentially of RHSiO-groups, R2ZSi0; groups and optionally R2SiO-groups and having a viscosity at 25° C. of no more than 1000 mm²/s, wherein R denotes an alkyl or aryl group having no more than 8 carbon atoms, and Z denotes H or R,
c. a diorganohydrogensiloxy-terminated polydiorganosiloxane, wherein the organic substituents are alkyl or aryl groups having no more than 8 carbon atoms,
d. a hydrosilylation catalyst for the reaction of SiH groups with Si-Alkenyl groups, wherein the vehicle Y is formulated in order that:
RHV>3 wherein RHV is the ratio of the number of SiH moles in (b) and (c) with respect to the number of Si-Alkenyl moles in (a) and (d), and
0<RHC<0.7 wherein RHC is the ratio of the number of SiH moles in (c) with respect to the number of SiH moles in (b) and (c).
